(19) United States
(12) Patent Application Publication
(10) Pub. No.: US 2019/0388035 A1
(43) Pub. Date: Dec. 26, 2019

(54) BLOOD PRESSURE DATA PROCESSING APPARATUS, BLOOD PRESSURE DATA PROCESSING METHOD, AND BLOOD PRESSURE DATA PROCESSING PROGRAM

(71) Applicant: OMRON HEALTHCARE CO., LTD., Muko-shi (JP)

(72) Inventors: Eriko KAN, Kyoto (JP); Hirotaka WADA, Kyoto (JP)

(73) Assignee: OMRON HEALTHCARE CO., LTD., Muko-shi, Kyoto (JP)

(21) Appl. No.: 16/561,734

(22) Filed: Sep. 5, 2019

Related U.S. Application Data
(63) Continuation of application No. PCT/JP2018/009585, filed on Mar. 12, 2018.

(30) Foreign Application Priority Data
Mar. 14, 2017 (JP) 2017-048932

(51) Int. Cl. A61B 5/00; A61B 5/021; A61B 5/11

(52) U.S. Cl. CPC A61B 5/721; A61B 5/021; A61B 5/1118; A61B 2562/0219; A61B 2562/0247; A61B 2562/046; A61B 5/6801

(57) ABSTRACT

A blood pressure data processing apparatus including a body movement index calculation unit, body movement intensity determination unit, and blood pressure data processing unit. The calculation unit calculates a body movement index of a user, which is a statistical value, in a unit period, of motion data obtained from a wearable motion sensor or pressure sensor data obtained from a pressure sensor array in a wearable blood pressure sensor. The determination unit determines which of a plurality of levels a user body movement intensity in the unit period corresponds to based on the body movement index. The blood pressure data processing unit performs first data processing on user blood pressure data obtained in the unit period if the body movement intensity is determined to be a first level, and performs second data processing if the body movement intensity is determined to be a second level.

### TECHNICAL FIELD

The present invention relates to processing blood pressure data.

### BACKGROUND ART

Patients with blood pressure disorders (typically, high blood pressure) desirably manage their blood pressure on a daily basis. Conventional stationary blood pressure measuring apparatuses are not suitable for users to carry around with them, and thus measuring blood pressure away from home such as at work or on an outing is a significant encumbrance for users. Also, steep fluctuations in blood pressure that can increase the onset risk of cerebrovascular and cardiovascular disease are extremely difficult to pick up on simply by measuring blood pressure several times a day.

In recent years, user terminals capable of measuring the blood pressure of a user simply by being worn on the user's wrist, for example, have been realized, following advances in sensor technology. Such user terminals enable blood pressure to be measured on a timely basis, without significantly encumbering users. There are also user terminals that are able to continuously measure blood pressure per beat, using techniques such as the tonometry method, for example.

If there is body movement in the user, the accuracy of blood pressure measurement may drop as a result of noise occurring due to the influence of the body movement or the like. In particular, in the case of continuously measuring the blood pressure of a user, the user may not always be in a resting state, and thus the blood pressure data may possibly include both reliable data and unreliable data. Thus, in the case of analyzing this blood pressure data, occurrence of the abovementioned steep fluctuations in blood pressure may be overlooked or, conversely, falsely detected.

### SUMMARY OF INVENTION

### Technical Problem

Discarding the data without exception is envisaged as a measure for dealing with blood pressure data containing noise due to body movement or the like. However, the influence of noise on blood pressure data is greater as the intensity of the body movement becomes stronger. In other words, if the intensity of the body movement is not that strong, the influence of noise on the blood pressure data is similarly not that great. Therefore, as a result of this measure, even blood pressure data that is not that greatly affected by noise is discarded and can no longer be used in analysis. Suppressing frequency components in which noise could possibly occur from blood pressure data is also envisaged as another measure. However, because body movement noise can be distributed over a wide frequency band, particularly in blood pressure data obtained through continuous measurement, this measure might result in required information (blood pressure fluctuations due to biological reaction) being impaired.

### Solution to Problem

An object of the present invention is to effectively reduce noise contained in blood pressure data.

According to a first aspect of the present invention, a blood pressure data processing apparatus includes a body movement index calculation unit, a body movement intensity determination unit, and a blood pressure data processing unit. The body movement index calculation unit is configured to calculate a body movement index which is a statistical value, in a unit period, of motion data obtained from a motion sensor worn by a user or pressure sensor data obtained from a pressure sensor array included in a blood pressure sensor worn by the user. The body movement intensity determination unit is configured to determine which of a plurality of levels including a first level and a second level a body movement intensity of the user in the unit period corresponds to based on the body movement index. The blood pressure data processing unit is configured to perform first data processing on blood pressure data obtained from the user in the unit period if the body movement intensity is determined to be the first level, and to perform second data processing on the blood pressure data if the body movement intensity is determined to be the second level. Therefore, noise can be effectively reduced by performing data processing suitable for the noise contained in blood pressure data.

According to a second aspect of the present invention, the body movement intensity determination unit determines that the body movement intensity is the first level if the body movement index is greater than or equal to a first threshold and less than a second threshold that is larger than the first threshold, and determines that the body movement intensity is the second level if the body movement index is greater than or equal to the second threshold. Therefore, noise can be effectively reduced by performing data processing suitable for the intensity of the noise contained in blood pressure data.

According to a third aspect of the present invention, the first data processing is processing for smoothing blood pressure data of a first period. The second data processing is processing for replacing blood pressure data with blood pressure data generated through interpolation based on blood pressure data before and after the unit period. Therefore, the first data processing is able to suppress (high frequency) noise that has little effect on blood pressure while maintaining the basic fluctuation components of the blood pressure data, and the second data processing is able to discard unreliable blood pressure data and utilize blood pressure data generated utilizing temporal correlation of the blood pressure data.

According to a fourth aspect of the present invention, the first threshold and the second threshold are determined based on a distribution of the body movement index calculated based on motion data obtained from a motion sensor or pressure sensor data obtained from a pressure sensor array included in a blood pressure sensor worn by the user or another user. Therefore, the intensity of the body movement can be determined with high accuracy.

According to a fifth aspect of the present invention, the second threshold is determined so as to be greater than or equal to an upper limit of a normal value statistically calculated from the distribution. Therefore, in the case where a body movement index corresponding to a high value (outlier) that greatly diverges from other values in the distribution is obtained, the body movement intensity can be determined to be the second level.

According to a sixth aspect of the present invention, the first threshold and the second threshold are determined based on an attribute of the user or an attribute of an environment in which blood pressure is measured. Therefore, the body movement intensity can be determined with high accuracy, with consideration for the influence by attributes of the user or attributes of the environment in which blood pressure is measured.

According to a seventh aspect of the present invention, the body movement intensity determination unit, in a case where the body movement intensity is determined to be the second level, further determines whether a continuous time for which the body movement intensity is determined to be the second level is longer than a third threshold. The blood pressure data processing unit performs the second data processing on the blood pressure data if the body movement intensity is determined to be the second level and the continuous time is determined to be less than or equal to the third threshold, and performs third data processing on the blood pressure data if the continuous time is determined to be longer than the third threshold. Therefore, more appropriate data processing can be determined by focusing not only on the body movement intensity of the user in the unit period but also on the continuous time for which that body movement intensity is determined.

According to an eighth aspect of the present invention, the second data processing is processing for replacing the blood pressure data with data generated through interpolation based on blood pressure data before and after the unit period. The third data processing is processing for discarding blood pressure data spanning the continuous time. Therefore, the second data processing is able to discard unreliable blood pressure data, and utilize blood pressure data generated utilizing temporal correlation of blood pressure data, and the third data processing is able to discard unreliable blood pressure data that spans a long period of time and is unsuitable for generation of blood pressure data by the second data processing.

According to a ninth aspect of the present invention, the unit period is determined so as to substantially coincide with one cycle or a plurality of cycles of a beat. Therefore, data processing for noise reduction can be performed in beat units.

According to a tenth aspect of the present invention, the blood pressure sensor is a blood pressure sensor that uses a tonometry method. Therefore, a body movement index that is based on tonogram data can be obtained.

According to an eleventh aspect of the present invention, the body movement index calculation unit calculates a plurality of body movement indices. The body movement intensity determination unit determines which of the plurality of levels the body movement intensity of the user in the unit period corresponds to based on the plurality of body movement indices. Therefore, the body movement intensity can be determined with high accuracy.

### Advantageous Effects of Invention

According to the present invention, noise that is contained in blood pressure data can be effectively reduced.

### BRIEF DESCRIPTION OF DRAWINGS

FIG. 1 is a block diagram illustrating a blood pressure data processing apparatus according to a first embodiment.

FIG. 2 is a flowchart illustrating operations of the blood pressure data processing apparatus of FIG. 1.

FIG. 3 is an illustrative diagram of first data processing that is performed by a first data processing unit of FIG. 1.

FIG. 4 is an illustrative diagram of the first data processing that is performed by the first data processing unit of FIG. 1.

FIG. 5 is an illustrative diagram of second data processing performed by a second data processing unit of FIG. 1.

FIG. 6 is an illustrative diagram of the second data processing performed by the second data processing unit of FIG. 1.

FIG. 7 is a block diagram illustrating a blood pressure data processing apparatus according to a second embodiment.

FIG. 8 is a flowchart illustrating operations of the blood pressure data processing apparatus of FIG. 7.

### DESCRIPTION OF EMBODIMENTS

Hereinafter, description of embodiments will be given with reference to the drawings. Note that, below, the same or similar reference numerals are given to elements that are the same or similar to elements that have already been described, and redundant description will generally be omitted.

### First Embodiment

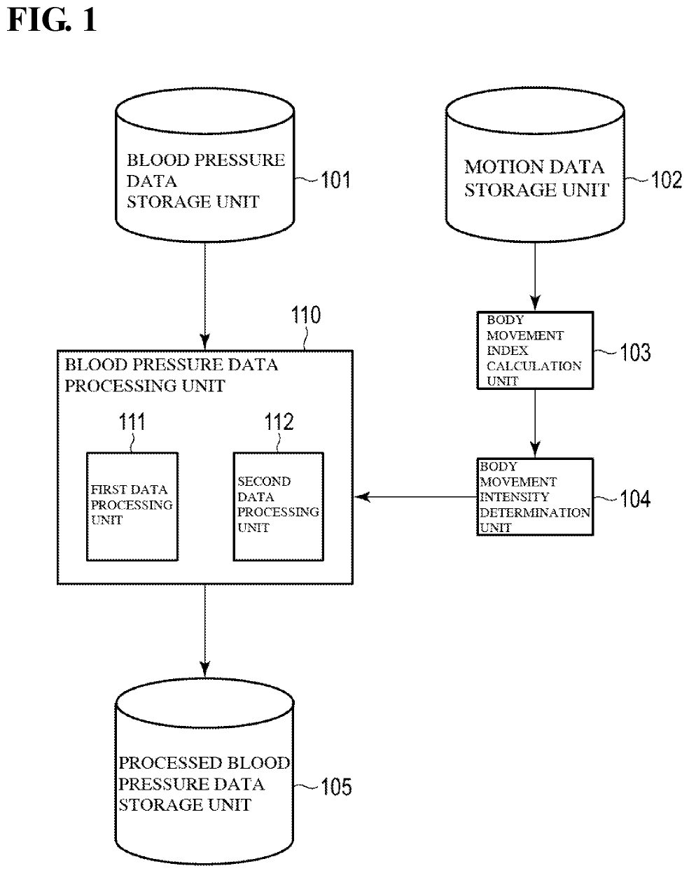

As illustrated in FIG. 1, a blood pressure data processing apparatus according to a first embodiment includes a blood pressure data storage unit 101, a motion data storage unit 102, a body movement index calculation unit 103, a body movement intensity determination unit 104, a processed blood pressure data storage unit 105, and a blood pressure data processing unit 110.

The blood pressure data storage unit 101 stores blood pressure data obtained by performing measurement (e.g., continuous measurement) of blood pressure with a blood pressure sensor worn by the user. The blood pressure data stored in the blood pressure data storage unit 101 is read out by the blood pressure data processing unit 110 as needed.

The blood pressure data can, for example, include the values of systolic blood pressure and diastolic blood pressure per beat, but is not limited thereto. The blood pressure data can be associated with respective measurement times.

The blood pressure sensor worn by the user can include a blood pressure sensor (referred to below as “continuous blood pressure sensor”) that is capable of continuously measuring the blood pressure of the user per beat. Continuous blood pressure sensors may continuously measure the blood pressure of the user from the pulse transit time (PTT), or may realize continuous measurement by the tonometry method or other techniques.

The blood pressure sensor can, in addition to a continuous blood pressure sensor, also include a blood pressure sensor (referred to below as “discontinuous blood pressure sensor”) that is not capable of continuous measurement. Discontinuous blood pressure sensors measure the blood pressure of the user using a cuff as the pressure sensor (oscillometric method), for example.

Discontinuous blood pressure sensors (particularly blood pressure sensors using the oscillometric method) tend to have high measurement accuracy, compared with continuous blood pressure sensors. Therefore, the blood pressure sensor may, for example, measure blood pressure data with higher accuracy, by operating the discontinuous blood pressure sensor instead of the continuous blood pressure sensor, triggered by some sort of condition being met (e.g., blood pressure data of user measured by continuous blood pressure sensor indicating a predetermined high risk state).

The motion data storage unit 102 stores motion data obtained by measuring motion with a motion sensor worn by the user. The motion data stored in the motion data storage unit 102 is read out by the body movement index calculation unit 103 as needed.

Motion data can, for example, include the values of one or a plurality of axes of acceleration or angular velocity, but is not limited thereto. Blood pressure data can be associated with respective measurement times. The motion sensor may be an acceleration sensor or an angular velocity sensor, for example. As an example, the motion sensor may be a triaxial acceleration sensor.

The body movement index calculation unit 103 reads out motion data from the motion data storage unit 102. The body movement index calculation unit 103 calculates a statistical value of the motion data in a unit period. This statistical value will be referred to as a body movement index, given that it is used in the determination of body movement intensity discussed later. The body movement index calculation unit 103 outputs the body movement index to the body movement intensity determination unit 104.

Here, the unit period may, for example, be the interval between consecutive beats, that is, one beat cycle (e.g., interval from start point to end point). Alternatively, the unit period may be a plurality of connected intervals, that is, a plurality of beat cycles. Data processing for noise reduction can thereby be performed per beat.

This body movement index may, for example, be (a) the mean value, standard deviation (SD), root-mean square (RMS) or range of a component value of an arbitrary axis (X-axis, Y-axis or Z-axis) of acceleration data in a unit period, or the slope of a single regression line based on the component value; (b) the maximum value, mean value, range, SD or total value of the difference absolute value between a triaxial composite value of acceleration data in a unit period and a reference 1G; (c) the mean value, SD, RMS, range of a triaxial composite value of acceleration data in a unit period or the slope of a single regression line based on the triaxial composite value; (d) the maximum range value, mean value, range, RMS, SD or total value of a component value of each axis (X-axis, Y-axis and Z-axis) of acceleration data in a unit period; (e) the maximum value, mean value, range, RMS, SD or total value of the slope of a single regression line based on a component value of each axis (X-axis, Y-axis and Z-axis) of acceleration data in a unit period; (f) the maximum mean value, mean value, range, RMS, SD or total value of a component value of each axis (X-axis, Y-axis and Z-axis) of acceleration data in a unit period; (g) the maximum RMS value, mean value, range, RMS, SD or total value of a component value of each axis (X-axis, Y-axis and Z-axis) of acceleration data in a unit period; or (h) the total SD value, maximum value, mean value, range, RMS or SD of a component value of each axis (X-axis, Y-axis and Z-axis) of acceleration data in a unit period, but is not limited thereto. Note that the body movement index calculation unit 103 may calculate a similar statistical value for angular velocity data as the body movement index instead of acceleration data.

Note that in the case where the blood pressure of the user is measured with a blood pressure sensor that uses the tonometry method, tonogram data obtained from a pressure sensor array included in the blood pressure sensor may be used instead of motion data. Because the pressing force of the pressure sensor changes with body movement, body movement can be estimated from this change. In this case, a tonogram data storage unit may be provided instead of or in addition to the motion data storage unit 102. Note that pressure sensor data obtained from a blood pressure sensor including a pressure sensor array that uses another method may be used instead of tonogram data as appropriate.

The body movement index calculation unit 103 reads out tonogram data from the tonogram data storage unit. The body movement index calculation unit 103 calculates the statistical value of the tonogram data in the unit period as the body movement index.

This body movement index may, for example, be (i) the amount of change in the output value of an active sensor (sensor selected for blood pressure calculation from a plurality of pressure sensors) between the start point (=t) and end point (=t+Δt, where Δt can be determined in one beat cycle or a plurality of beat cycles) of a unit period, (j) the square of a correlation coefficient or a normalized correlation coefficient of the output value of all pressure sensors at the start point of a unit period and the output value of all pressure sensors at the end point of the unit period; (k) the total amount of change of the output value of each pressure sensor at the start point of a unit period and the output value of each pressure sensor at the end point of the unit period; or (l) the difference in the output values of pressure sensors separated on either side of a peak sensor (sensor with maximum pressure value) by a predetermined number of pressure sensors, but is not limited thereto.

The body movement intensity determination unit 104 receives the body movement index from the body movement index calculation unit 103. The body movement intensity determination unit 104 determines which of a plurality of levels including a first level (value “1”) and a second level (value “2”) the body movement intensity of the user in the unit period corresponds to based on the body movement index. Note that the number of levels that can be determined by the body movement intensity determination unit 104 is not limited to two, and may be three or more. The body movement intensity determination unit 104 notifies the determined body movement intensity to the blood pressure data processing unit 110.

For example, the body movement intensity determination unit 104 determines that the body movement intensity is the first level (value “1”), if the body movement index is greater than or equal to a first threshold (Th1) and less than a second threshold (where Th2>Th1). The body movement intensity determination unit 104 determines that the body movement intensity is the second level (value “2”), if body movement index is greater than or equal to the second threshold (Th2).

The first threshold (Th1) and the second threshold (Th2) can be defined based on the distribution of the body movement index. This distribution is, for example, obtained by calculating the body movement index based on motion data obtained from a motion sensor or tonogram data obtained from a pressure sensor array included in a blood pressure sensor that uses the tonometry method worn by a user (can be not only the user whose body movement intensity is to be determined but another user). The distribution may use the motion data or tonogram data obtained when the user is at rest (e.g., asleep). Using the first threshold (Th1) and second threshold (Th2) determined using this distribution enables it to be determined whether a given body movement index is significantly large compared with the body movement index when the user is at rest.

The distribution of the body movement index may be provided for every attribute of the user (e.g., age, gender, condition, sleep state, active state, etc.) or every attribute of the environment in which the motion data or tonogram data was obtained (e.g., season, month, day-of-week, time, location, temperature, humidity, etc.). The first threshold (Th1) and second threshold (Th2) can thereby be determined according to an attribute of the user or an attribute of the environment in which the blood pressure of the user is measured. In this way, by making the determination threshold of the body movement intensity variable, the determination accuracy of the body movement intensity can be enhanced, thus enabling more appropriate data processing to be performed on the blood pressure data. That is, quality (low noise and blood pressure fluctuations due to biological reaction are maintained) processed blood pressure data is obtained. On the other hand, it is also possible to simplify processing by fixing the thresholds for determining body movement intensity.

The second threshold (Th2) can be used in order to determine values (outliers) that diverge greatly from other values in this distribution. As an example, the second threshold (Th2) may be determined so as to be greater than or equal to an upper limit of a normal value statistically calculated from the distribution. The upper limit of the normal value may be determined based on a third quartile which is a state of robust health relative to the outlier, for example. Specifically, the second threshold (Th2) may be determined so as to substantially coincide with the third quartile+1.5×IQR (interquartile range). Also, the first threshold (Th1) may be determined so as to substantially coincide with half of the second threshold (Th2).

Note that in the case where the body movement index calculation unit 103 calculates a plurality of body movement indices, the body movement intensity determination unit 104 may determine the body movement intensity based on the number or percentage of body movement indices that is greater than or equal to a reference value. A reference value may be determined for every body movement index, and a common reference value can also be used if the body movement indices are normalized.

The blood pressure data processing unit 110 reads out blood pressure data spanning the unit period from the blood pressure data storage unit 101. The blood pressure data processing unit 110 determines the data processing to be applied to this blood pressure data, based on the body movement intensity determined for this unit period. The blood pressure data processing unit 110 performs the determined data processing on the blood pressure data to generate processed blood pressure data, and saves the processed blood pressure data to the processed blood pressure data storage unit 105. Note that if it is determined that the body movement intensity is also less than the first level (e.g., body movement index is less than the first threshold (Th1)), the blood pressure data need not be processed (pass through).

Specifically, the blood pressure data processing unit 110 respectively performs first data processing or second data processing on blood pressure data if the body movement intensity is the first level (value “1”) or the second level (value “2”). Processing that has a strong noise suppression effect compared to the first data processing is employed for the second data processing.

The blood pressure data processing unit 110 includes a first data processing unit 111 for performing first data processing and a second data processing unit 112 for performing second data processing.

The first data processing unit 111 performs the first data processing on the blood pressure data of a unit period in which the body movement intensity is determined to be the first level (value “1”). Note that the first data processing unit 111 may perform the first data processing to also include the peripheral (e.g., ±n beats, where n is an arbitrary numerical value) blood pressure data of that unit period. The period in which the first data processing is performed may be variable. The first data processing is, for example, processing for smoothing target blood pressure data. The smoothing may be performed using a smoothing technique such as moving average smoothing, for example. According to this first data processing, (high frequency) noise that has little effect of blood pressure can be suppressed while maintaining the basic fluctuation components of the blood pressure data.

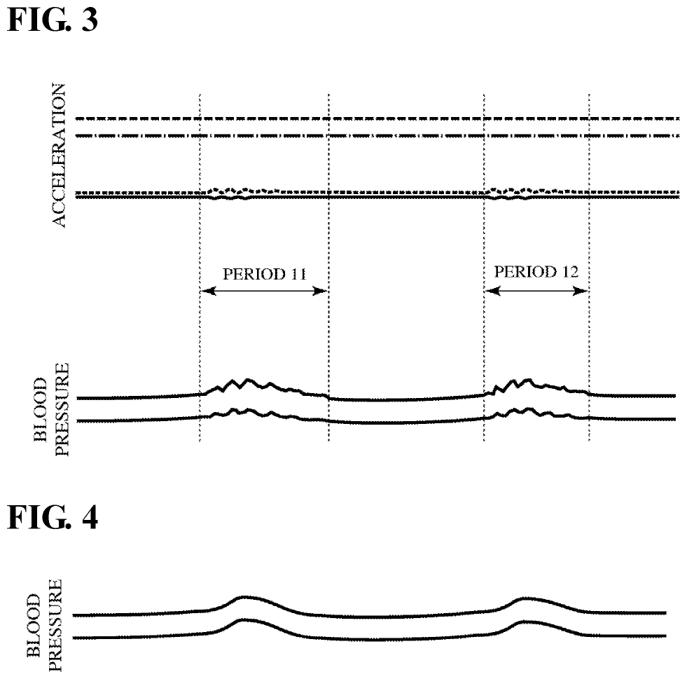

FIG. 3 illustrates acceleration data and blood pressure data. Assume that the body movement intensity in a period 11 and a period 12 of FIG. 3 is determined to be the first level (value “1”). In this case, the first data processing unit 111 smoothes the blood pressure data of the period 11 and the period 12, and can generate the processed blood pressure data illustrated in FIG. 4.

The second data processing unit 112 performs the second data processing on the blood pressure data of a unit period in which the body movement intensity is determined to be the second level (value “2”). The second data processing is, for example, processing for replacing target blood pressure data with blood pressure data generated through interpolation based on blood pressure data therebefore and thereafter. An interpolation method such as linear interpolation or spline interpolation, for example, may be used in generating blood pressure data through interpolation. According to this second data processing, unreliable blood pressure data can be discarded and blood pressure data generated utilizing temporal correlation of blood pressure data can be utilized. The period in which the second data processing is performed may coincide with the unit period or may be obtained by extending the unit period. Also, this period may be variable. Note that it takes longer for blood pressure to return to a normal state as the body movement intensity increases. Therefore, the length of this period may, for example, be adjusted so as to be longer as the body movement index becomes higher.

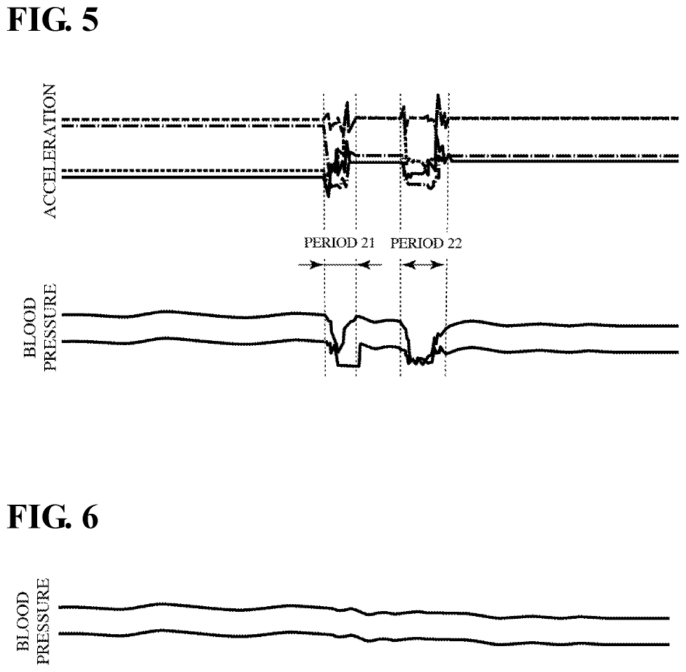

FIG. 5 illustrates acceleration data and blood pressure data. Assume that the body movement intensity in a period 21 and a period 22 of FIG. 5 is determined to be the second level (value “2”). In this case, the second data processing unit 112 replaces the blood pressure data in the period 21 and the period 22 with blood pressure data generated through interpolation based on respective blood pressure data therebefore and thereafter, and can generate the processed blood pressure data illustrated in FIG. 6.

The processed blood pressure data storage unit 105 stores the processed blood pressure data. This processed blood pressure data may be read out as needed by a functional unit or apparatus for blood pressure data processing that is not illustrated in order to detect steep fluctuations in blood pressure, for example.

A steep fluctuation in blood pressure refers, for example, to a steep fluctuation in blood pressure that may be triggered by hypoxia in sleep apnea syndrome (SAS) during sleep. Accordingly, monitoring the frequency of steep fluctuations in blood pressure is useful in ascertaining the severity of symptoms of SAS in the user.

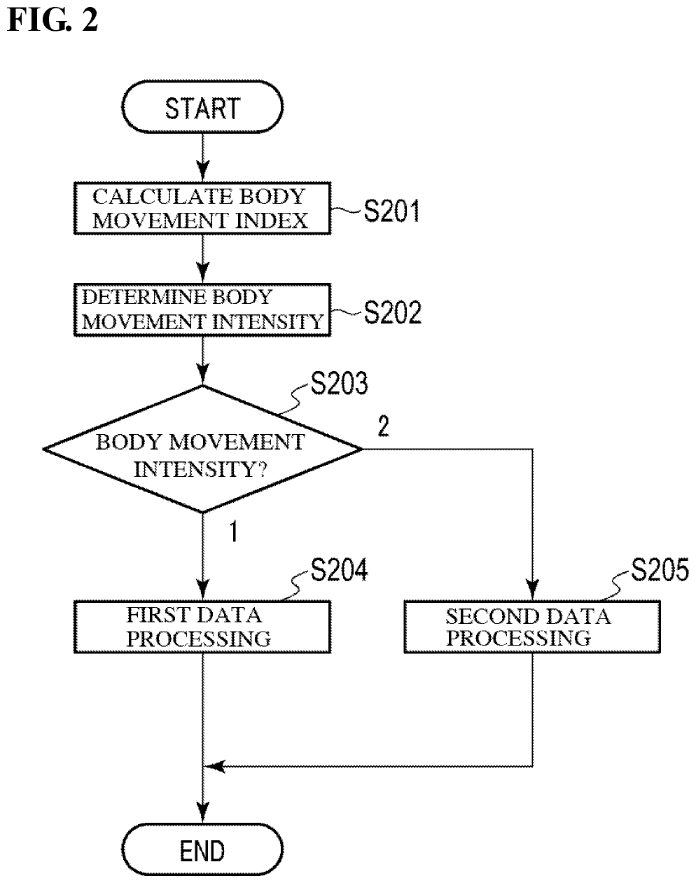

The blood pressure data processing apparatus of FIG. 1 operates as illustrated in FIG. 2. The operations of FIG. 2 may be periodically implemented every unit period, for example, or may be collectively performed for a plurality of unit periods.

In step S201, the body movement index calculation unit 103 reads out motion data from the motion data storage unit 102, and calculates a body movement index which is a statistical value of the motion data in the unit period. Note that, in step S201, the body movement index calculation unit 103 may calculate the body movement index using tonogram data instead of motion data.

The body movement intensity determination unit 104 determines the body movement intensity of the unit period over three or more stages (three stages in the example in FIG. 2), by comparing the body movement index calculated in step S201 with a plurality of thresholds (step S203).

If the body movement intensity is determined to be the first level (value “1”) (step S203), the processing advances to step S204. If the body movement intensity is determined to be the second level (value “2”) (step S203), the processing advances to step S205.

In step S204, the first data processing unit 111 performs the first data processing on the blood pressure data of the unit period. In step S205, the second data processing unit 112 performs the second data processing on the blood pressure data of the unit period.

As described above, the blood pressure data processing apparatus according to the first embodiment determines the body movement intensity of the user in a unit period over at least three stages, and performs data processing associated with the determined body movement intensity on the blood pressure data of the user in the unit period. Specifically, this blood pressure data processing apparatus performs different data processing on blood pressure data measured when the body movement intensity is high and blood pressure data measured when the body movement intensity is moderate. Therefore, noise can be effectively reduced by performing data processing suitable for the noise (intensity) contained in the blood pressure data.

### Second Embodiment

The blood pressure data processing apparatus according to the first embodiment determines the data processing to be performed on the blood pressure data of a unit period depending on the body movement intensity of the user in the unit period. However, for example, when the body movement intensity is continually determined to be the second level (value “2”) across a plurality of consecutive unit periods, blood pressure data in the consecutive unit periods will be generated through interpolation from blood pressure data therebefore and thereafter. The validity of the blood pressure data generated through interpolation decreases as the number of continuous unit periods increases. Therefore, the blood pressure data processing apparatus according to a second embodiment determines more appropriate data processing by focusing not only on the body movement intensity of the user in the unit period but also on the continuous time for which that body movement intensity is determined.

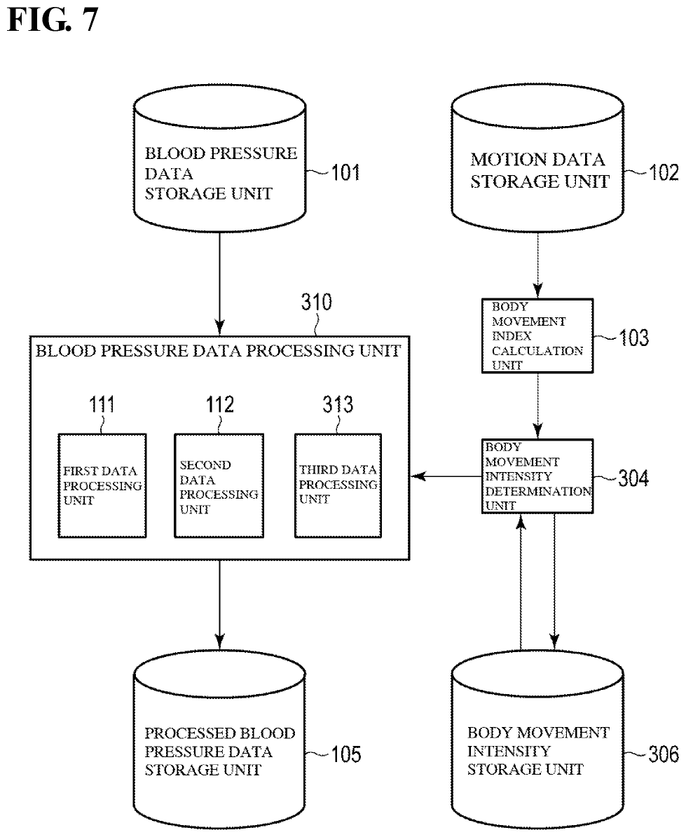

As illustrated in FIG. 7, the blood pressure data processing apparatus according to the second embodiment includes a blood pressure data storage unit 101, a motion data storage unit 102, a body movement index calculation unit 103, a body movement intensity determination unit 304, a processed blood pressure data storage unit 105, a body movement intensity storage unit 306, and a blood pressure data processing unit 310.

The body movement intensity determination unit 304 receives body movement index from the body movement index calculation unit 103. The body movement intensity determination unit 304 determines which of a plurality of levels including a first level (value “1”) and a second level (value “2”) the body movement intensity of the user in a unit period corresponds to based on body movement index. Note that the number of levels that can be determined by the body movement intensity determination unit 304 is not limited to three, and may be four or more.

The body movement intensity determination unit 304 stores the determined body movement intensity in the body movement intensity storage unit 306. The body movement intensity determination unit 304 may be configured to store the determined body movement intensity in the body movement intensity storage unit 306, only in the case where the determined body movement intensity is a specific level (e.g., second level (value “2”)). The body movement intensity determination unit 304, if the body movement intensity of the user in the unit period is the second level (value “2”), derives the continuous time for which the body movement intensity is determined to be the second level (value “2”), with reference to the body movement intensity storage unit 306. The body movement intensity determination unit 304 then further determines whether this continuous time is longer than a third threshold. The body movement intensity determination unit 304 notifies the determined body movement intensity and, in the case where this body movement intensity is the second level (value “2”), the determination result as to whether that continuous time is longer than the third threshold to the blood pressure data processing unit 310.

The blood pressure data processing unit 310 reads out the blood pressure data spanning the unit period from the blood pressure data storage unit 101. The blood pressure data processing unit 310 determines the data processing to be applied to this blood pressure data, based on the body movement intensity determined for this unit period and, in the case where this body movement intensity is the second level (value “2”), the determination result as to whether the continuous time thereof is longer than the third threshold. The blood pressure data processing unit 310 performs the determined data processing on the blood pressure data, generates processed blood pressure data, and saves this processed blood pressure data to the processed blood pressure data storage unit 105.

Specifically, the blood pressure data processing unit 310 performs first data processing on the blood pressure data, if the body movement intensity is the first level (value “1”). The blood pressure data processing unit 310, in the case where the body movement intensity is the second level (value “2”), performs second data processing on the blood pressure data if the continuous time is less than or equal to the third threshold, and perform third data processing on the blood pressure data if the continuous time is longer than the third threshold. Note that processing need not be performed on the blood pressure data (pass through), if it is determined that the body movement intensity is less than the first level (e.g., body movement index is less than the first threshold (Th1)).

The blood pressure data processing unit 310 includes a first data processing unit 111 for performing the first data processing, a second data processing unit 112 for performing the second data processing, and a third data processing unit 313 for performing the third data processing.

The third data processing unit 313 performs the third data processing on the blood pressure data spanning the continuous time for which the body movement intensity is determined to be the second level (value “2”). The third data processing is, for example, processing for discarding the target blood pressure data. According to this third data processing, unreliable blood pressure data that spans a long period of time and is unsuitable for generation of blood pressure data by the second data processing can be discarded.

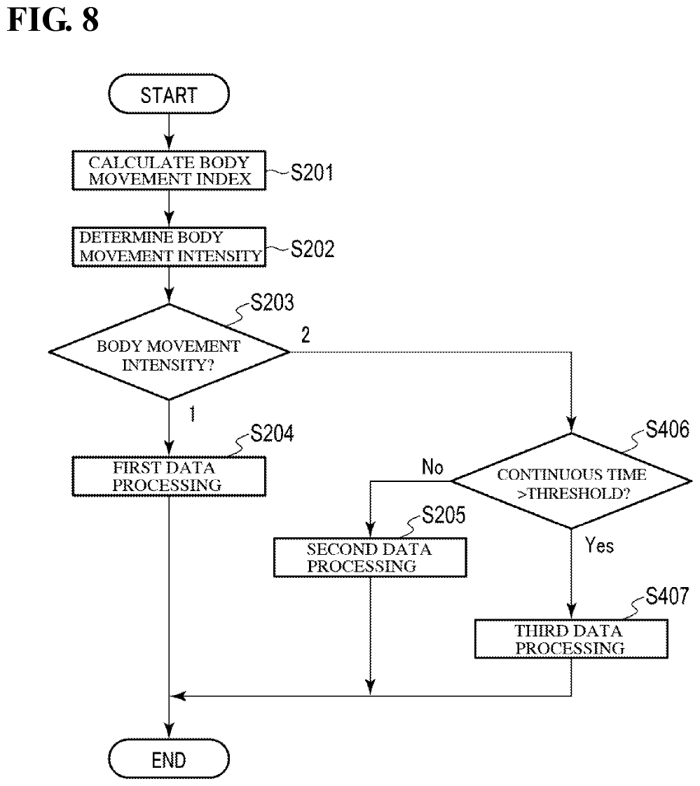

The blood pressure data processing apparatus of FIG. 7 operates as illustrated in FIG. 8. The operations of FIG. 8 may, for example, be periodically implemented every unit period, or may be collectively performed on a plurality of unit periods. The operations of FIG. 8 are similar to the operations of FIG. 2 with respect to the processing performed in steps S201 to S205. In step S203 of FIG. 8, however, in the case where the body movement intensity is determined to be the second level (value “2”), the processing advances to step S406 rather than step S205.

In step S406, the body movement intensity determination unit 304 determines whether the continuous time for which the body movement intensity is determined to be the second level (value “2”) is longer than the third threshold. If the continuous time is longer than the third threshold, the processing advances to step S407, and if this is not the case, the processing advances to step S205. In step S407, the third data processing unit 113 performs the abovementioned third data processing on the blood pressure data spanning the continuous time.

As described above, the blood pressure data processing apparatus according to the second embodiment further evaluates the length of the continuous time for which the body movement intensity is determined to be a specific level, and performs data processing that differs depending on whether this continuous time is long or short. Specifically, this blood pressure data processing apparatus performs generation through interpolation in the case where the continuous time is short, but discards the blood pressure data in the case where the continuous time is long and supplementing data by generating blood pressure data through interpolation is not appropriate. Therefore, noise can be effectively reduced by performing data processing suitable for the intensity and continuous time of the noise that is contained in blood pressure data.

The above embodiments merely show examples for aiding understanding of the concepts of the present invention, and are not intended to limit the scope of the invention. In the embodiments, addition, deletion or substitution of various constituent elements can be carried out without departing from the spirit of the invention.

Various functional units described in the above embodiments may be realized by using a circuit. The circuit may be a dedicated communication circuit that realizes a specific function, or may be a general-purpose circuit such as a processor.

The processing of the above embodiments can be at least partially realized by using a general-purpose computer as basic hardware. Programs that realize the above processing may be provided by being stored on a computer-readable recording medium. Programs are stored on the recording medium as installable files or executable files. Exemplary recording media include magnetic disk, optical disk (CD-ROM, CD-R, DVD, etc.), magneto-optical disk (MO, etc.), and semiconductor memory. Any recording medium that is able to store programs and is readable by computer may be applied. Also, programs that realize the above processing may be stored on a computer (server) connected to a network such as the Internet, and downloaded to a computer (client) via the network.

The above embodiments can be partially or wholly described as in the following supplementary note, apart from the claims, but are not restricted thereto.

### Supplementary Note 1

A blood pressure data processing apparatus including:
a memory; and
a processor connected to the memory,
the processor being configured to:
(a) calculate a body movement index which is a statistical value, in a unit period, of motion data obtained from a motion sensor worn by a user or pressure sensor data obtained from a pressure sensor array included in a blood pressure sensor worn by the user;
(b) determine which of a plurality of levels including a first level and a second level a body movement intensity of the user in the unit period corresponds to based on the body movement index; and
(c) perform first data processing on blood pressure data obtained from the user in the unit period if the body movement intensity is determined to be the first level, and perform second data processing on the blood pressure data if the body movement intensity is determined to be the second level.

1. A blood pressure data processing apparatus comprising:
a body movement index calculation unit configured to calculate a body movement index which is a statistical value, in a unit period, of motion data obtained from a motion sensor worn by a user or pressure sensor data obtained from a pressure sensor array included in a blood pressure sensor worn by the user;
a body movement intensity determination unit configured to determine which of a plurality of levels including a first level and a second level a body movement intensity of the user in the unit period corresponds to based on the body movement index; and
a blood pressure data processing unit configured to perform first data processing corresponding to noise reduction processing or blood pressure data obtained from the user in the unit period if the body movement intensity is determined to be the first level, to perform second data processing corresponding to noise reduction processing on the blood pressure data if the body movement intensity is determined to be the second level, and to pass through the blood pressure data if the body movement intensity is determined to not satisfy the first level.

2. The blood pressure data processing apparatus according to claim 1,
wherein the body movement intensity determination unit determines that the body movement intensity is the first level if the body movement index is greater than or equal to a first threshold and less than a second threshold that is larger than the first threshold, and determines that the body movement intensity is the second level if the body movement index is greater than or equal to the second threshold.

3. The blood pressure data processing apparatus according to claim 2,
wherein the first data processing is processing for smoothing blood pressure data of a first period that encompasses the unit period, and
the second data processing is processing for replacing blood pressure data of a second period that encompasses the unit period with blood pressure data generated through interpolation based on blood pressure data before and after the second period.

4. The blood pressure data processing apparatus according to claim 2,
wherein the first threshold and the second threshold are determined based on a distribution of the body movement index calculated based on motion data obtained from a motion sensor or pressure sensor data obtained from a pressure sensor array included in a blood pressure sensor worn by the user or another user.

5. The blood pressure data processing apparatus according to claim 4,
wherein the second threshold is determined so as to be greater than or equal to an upper limit of a normal value statistically calculated from the distribution.

6. The blood pressure data processing apparatus according to claim 2,
wherein the first threshold and the second threshold are determined based on an attribute of the user or an attribute of an environment in which blood pressure is measured.

7. The blood pressure data processing apparatus according to claim 2,
wherein the body movement intensity determination unit, in a case where the body movement intensity is determined to be the second level, further determines whether a continuous time for which the body movement intensity is determined to be the second level is longer than a third threshold, and
the blood pressure data processing unit performs the second data processing on the blood pressure data if the body movement intensity is determined to be the second level and the continuous time is determined to be less than or equal to the third threshold, and performs third data processing on the blood pressure data if the continuous time is determined to be longer than the third threshold.

8. The blood pressure data processing apparatus according to claim 7,
wherein the second data processing is processing for replacing the blood pressure data with data generated through interpolation based on blood pressure data before and after the unit period, and
the third data processing is processing for discarding blood pressure data spanning the continuous time.

9. The blood pressure data processing apparatus according to claim 1,
wherein the unit period is determined so as to substantially coincide with one cycle or a plurality of cycles of a beat.

10. The blood pressure data processing apparatus according to claim 1,
wherein the blood pressure sensor is a blood pressure sensor that uses a tonometry method.

11. The blood pressure data processing apparatus according to claim 1,
wherein the body movement index calculation unit calculates a plurality of body movement indices, and
the body movement intensity determination unit determines which of the plurality of levels the body movement intensity of the user in the unit period corresponds to based on the plurality of body movement indices.

12. A blood pressure data processing method comprising:
calculating a body movement index which is a statistical value, in a unit period, of motion data obtained from a motion sensor worn by a user or pressure sensor data obtained from a pressure sensor array included in a blood pressure sensor worn by the user;
determining which of a plurality of levels including a first level and a second level a body movement intensity of the user in the unit period corresponds to based on the body movement index; and
performing first data processing corresponding to noise reduction processing on blood pressure data obtained from the user in the unit period if the body movement intensity is determined to be the first level, performing second data processing corresponding to noise reduction processing on the blood pressure data if the body movement intensity is determined to be the second level, and passing through the blood pressure data if the body movement intensity is determined to not satisfy the first level.

13. A blood pressure data processing program for causing a computer to function as the blood pressure data processing apparatus according to claim 1.

14. A blood pressure data processing apparatus comprising:
a body movement index calculation unit configured to calculate a body movement index which is a statistical value, in a unit period, of motion data obtained from a motion sensor worn by a user or pressure sensor data obtained from a pressure sensor array included in a blood pressure sensor worn by the user;
a body movement intensity determination unit configured to determine which of a plurality of levels including a first level and a second level a body movement intensity of the user in the unit period corresponds to based on the body movement index; and
a blood pressure data processing unit configured to perform first data processing on blood pressure data obtained from the user in the unit period if the body movement intensity is determined to be the first level, and to perform second data processing on the blood pressure data if the body movement intensity is determined to be the second level,
wherein the body movement intensity determination unit determines that the body movement intensity is the first level if the body movement index is greater than or equal to the first threshold and less than a second threshold that is larger than the first threshold, and determines that the body movement intensity is the second level if the body movement index is greater than or equal to the second threshold, and
the first threshold and the second threshold are determined based on an attribute of the user or an attribute of an environment in which blood pressure is measured.

15. A blood pressure data processing apparatus comprising:
a body movement index calculation unit configured to calculate a body movement index which is a statistical value, in a unit period, of motion data obtained from a motion sensor worn by a user or pressure sensor data obtained from a pressure sensor array included in a blood pressure sensor worn by the user;
a body movement intensity determination unit configured to determine which of a plurality of levels including a first level and a second level a body movement intensity of the user in the unit period corresponds to based on the body movement index; and
a blood pressure data processing unit configured to perform first data processing on blood pressure data obtained from the user in the unit period if the body movement intensity is determined to be the first level, and to perform second data processing on the blood pressure data if the body movement intensity is determined to be the second level,
wherein the body movement intensity determination unit determines that the body movement intensity is the first level if the body movement index is greater than or equal to the first threshold and less than a second threshold that is larger than the first threshold, and determines that the body movement intensity is the second level if the body movement index is greater than or equal to the second threshold,
the body movement intensity determination unit, in a case where the body movement intensity is determined to be the second level, further determines whether a continuous time for which the body movement intensity is determined to be the second level is longer than a third threshold, and
the blood pressure data processing unit performs the second data processing on the blood pressure data if the body movement intensity is determined to be the second level and the continuous time is determined to be less than or equal to the third threshold, and performs third data processing on the blood pressure data if the continuous time is determined to be longer than the third threshold.

16. The blood pressure data processing apparatus according to claim 15,
wherein the second data processing is processing for replacing the blood pressure data with data generated through interpolation based on blood pressure data before and after the unit period, and
the third data processing is processing for discarding blood pressure data spanning the continuous time.
